(19) United States
(12) Patent Application Publication
(10) Pub. No.: US 2005/0033405 A1
(43) Pub. Date: Feb. 10, 2005

(54) Rail stent-graft for repairing abdominal aortic aneurysm

(75) Inventor: Kenneth S. Solovay, Weston, FL (US)

Correspondence Address: GMP COMPANIES, INC., ONE EAST BROWARD BLVD., SUITE 1701, FORT LAUDERDALE, FL, 33301, US

(73) Assignee: GMP/Cardiac Care, Inc., Fort Lauderdale, FL (US)

(21) Appl. No.: 10/713,855

(22) Filed: Nov. 14, 2003

Related U.S. Application Data
(63) Continuation-in-part of application No. 10/641,284, filed on Aug. 15, 2003, now Pat. No. 6,805,706.
(60) Provisional application No. 60/403,361, filed on Aug. 15, 2002.
(60) Provisional application No. 60/426,420, filed on Nov. 15, 2002.

(51) Int. Cl. A61F 2/06

(52) U.S. Cl. 623/1.13; 623/1.16

(57) ABSTRACT

A rail stent-graft with increased longitudinal flexibility and sealing properties that is deployable within a body lumen, such as the aorta, for repairing an aneurysm. The rail stent-graft includes an anchoring assembly for securing the rail stent-graft to a portion of the vessel above a junction with another vessel.

### CROSS-REFERENCES TO RELATED APPLICATIONS

This application is a continuation-in-part of U.S. patent application Ser. No. 10/641,284 filed on Aug. 15, 2003, which claims the benefit of U.S. Provisional Patent Application No. 60/403,361 filed on Aug. 15, 2002, and this application also claims the benefit of U.S. Provisional Patent Application No. 60/426,420, filed on Nov. 15, 2002. The full disclosure of each of these applications is incorporated herein by reference.

### FIELD OF THE INVENTION

The present invention relates to a stent-graft for use as a prosthetic within a body vessel to support the vessel, and particularly, to a stent-graft having improved longitudinal structural flexibility and graft wear that can be used within a body vessel such as the aorta to support and facilitate the repair of the vessel.

### BACKGROUND OF THE INVENTION

An abdominal aortic aneurysm (AAA) is a very common deteriorating disease typically manifested by a bulbous weakened section and expansion of the aorta vessel wall at a region between the aorto-renal junction and the aorto-iliac junction. These aneurysms can result from accidents, atherosclerosis, high blood pressure or inherited disease. Aneurysms affect the ability of the vessel lumen to conduct fluids, and may at times be life threatening, for instance when rupture of the vessel wall occurs. Ruptured abdominal aortic aneurysms—which can cause massive internal bleeding—kill about 6,000 Americans a year.

A traditional treatment for repairing an aneurysm is to surgically remove part or all of the aneurysm and replace it with a synthetic graft or patch. But, in this procedure, the graft is put in place by threading a tiny plastic tube through a small incision in the groin and into a femoral artery. A spring-loaded stent graft, covered with a sheath, is loaded on the tip of the tube. The stent graft provides a new, more secure channel for blood within the blood vessel. Using X-ray images, the medical team guides the graft to the diseased section of the blood vessel and then pulls back the sheath. The self-expanding spring action fixes the graft to the inside vessel wall, and the tube is withdrawn from the femoral artery and the groin.

When the aneurysm is proximate the opening of another vessel, such as the renal artery, it can be difficult to anchor a conventional expandable stent-graft within the aorta. Additionally, the neck above the aneurysm can be short and tortuous. Conventional expandable stent-grafts may include anchors such as that disclosed in U.S. Pat. No. 6,334,869 to Leonhardt et al., which is incorporated herein by reference. These conventional, expandable stent-grafts with anchoring stent portions are commonly referred to as “suprarenal” stent-grafts. However, these suprarenal stent grafts do not conform to, or follow, the contour of the region of the aneurysm. As a result, these conventional tubular stent grafts can be too stiff for effective use at the site of an aortic aneurysm.

In cases where the aneurysm involves the ipsilateral and contralateral iliac vessels extending from the aorta, it is known to provide a generally Y-shaped bifurcated stent graft having a primary limb joining with an ipsilateral limb and a contralateral limb. An example of such a stent graft, and elements for surgically implanting the stent graft, are described in U.S. Pat. No. 5,387,235 to Chuter, which is incorporated herein by reference. The surgical procedure taught by Chuter involves either surgical isolation of the femoral vessels in the groin to provide direct access to the vessels, or percutaneous entry through both ipsilateral and contralateral femoral arteries. However, these stent grafts experience the same lack of longitudinal flexion that are experienced by the above-discussed conventional stent grafts.

### SUMMARY OF THE INVENTION

The present invention relates to a stent-graft with increased longitudinal flexibility relative to conventional stent-grafts. Longitudinal flexibility as used herein relates to the flexibility of the stent-graft structure (or portions thereof) to move relative to its major, longitudinal axis of extension. The stent-graft is deployed within a body lumen such as the aorta for supporting the lumen and repairing luminal aneurysms. In a preferred embodiment, the stent-graft is located and expanded within a blood vessel to repair aortic aneurysms.

An aspect of the present invention includes a rail stent-graft comprising an elongated stent assembly including at least one vessel support element that is positionable on a first side of a junction of at least two vessels. The rail stent-graft also includes an elongated stent-graft assembly comprising at least one vessel support element and at least one graft element. The stent-graft assembly is positionable on a second side of the junction of the at least two vessels. The rail stent-graft assembly further includes at least one rail element extending between the stent assembly and the stent-graft assembly. Each of these assemblies is moveable along and relative to the at least one rail element.

### BRIEF DESCRIPTION OF THE DRAWINGS

The present invention will be even better understood with reference to the attached drawings, in which:

FIG. 1 is a partial schematic illustration of a descending aorta;

FIG. 2 illustrates a rail stent-graft according to an embodiment of the present invention positioned with a descending aorta; and

FIG. 3 is a schematic view of the rail stent-graft shown in FIG. 2.

### DETAILED DESCRIPTION OF THE INVENTION

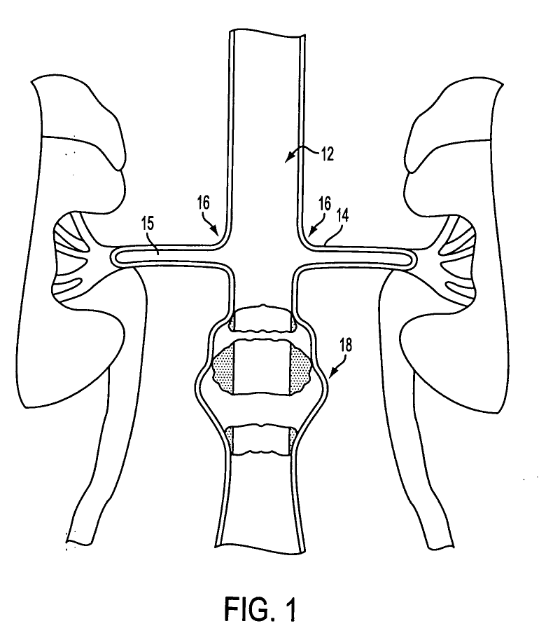

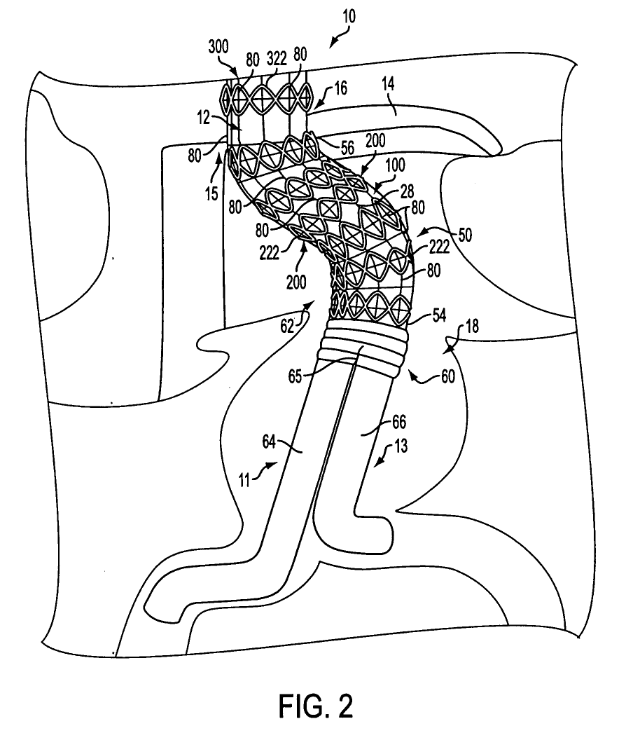

Referring to the figures where like numerals indicate the same element throughout the views, FIG. 1 shows an aorta 12 joined to renal arteries 14 and 15 at aorto-renal junctions (intersection) 16, and having an aortic aneurysm 18 below the aorto-renal junctions 16. As is known, an aortic aneurysm 18 includes a weakened and expanded vessel wall at the diseased region of the aorta 12. As shown in FIG. 2, the rail stent-graft 10 according to the present invention is deployed within the aorta 12 so that at least a stent-graft assembly 50 is located in the region of the aneurysm 18 and acts as a prosthetic device for relieving blood flow pressure against the weakened vessel wall by acting as a fluid conduit through the region of the aneurysm 18.

As illustrated in FIG. 2, the rail stent-graft 10 according to the present invention comprises the stent-graft assembly 50 including a graft portion 100 and a stent portion 200. The stent-graft assembly 50 can include the structure of the stent-grafts discussed in U.S. patent application Ser. No. 10/641,284 filed on Aug. 15, 2003, which is fully incorporated herein by reference. The rail stent-graft 10 also comprises a rail stent assembly 300 that is spaced from the stent-graft assembly 50 so that these two assemblies can be positioned on opposite sides of an intersection of two vessels. The rail stent assembly 300 can include any of the rail stents discussed in U.S. patent application Ser. No. 10/100,986 filed on Mar. 20, 2002, and U.S. Provisional patent application Ser. No. 60/426,366, filed on Nov. 15, 2002, which are both fully incorporated herein by reference. As illustrated, the rail stent assembly 300 can be positioned and anchored above the junction 16 in order to locate the assemblies 50, 300 of the rail stent-graft 10 at their respective desired positions within the aorta 12. As discussed below, elongated rail elements 80 extend between the assemblies 50 and 300. Any number of rails 80 that do not hinder the desired longitudinal flexibility of the stent-graft 10 can be used between the assemblies 50, 300 and within these assemblies 50, 300.

The stent portion 200 of the stent-graft assembly 50 includes a plurality of spaced, circumferential support elements (hoops) 222. Each circumferential support element 222 is generally annular in shape. In a preferred embodiment, each circumferential support element 222 has a sinusoidal or otherwise undulating form. Each circumferential support element 222 is made from a flexible, biocompatible material (i.e., from a material that is, for example, non-reactive and/or non-irritating). In one embodiment, each circumferential support element 222 is made from medical-grade metal wire formed as a closed loop (i.e., as an annular hoop) in a known manner, including, for example, micro-welding two ends of a wire segment together. Stainless steel, metal alloys, shape-memory alloys, super elastic alloys and polymeric materials used in conventional stents are representative examples of materials from which circumferential support elements 222 can be formed. The alloys can include NiTi and Nitinol. The polymers for circumferential support elements 222 may, for example, be bioabsorbable polymers so that the stent can be absorbed into the body instead of being removed.

As shown in FIG. 2, the support elements 222 are freely mounted on elongated rails elements 80 (herein after “rails”) such that the support elements 222 can move along the rails 80. The rails 80 extend along the length of the stent-graft 10 between the outermost peaks of terminal support elements 222 at a first end 54 and the innermost peaks of the terminal support element 222 at a second end 56. As illustrated, the terminal support elements 222 can extend beyond the terminal ends of the graft-portion 100.

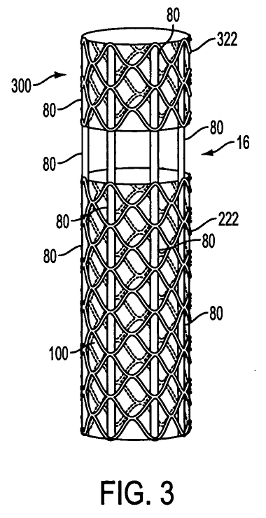

The graft portion 100, illustrated in FIGS. 2 and 3, is formed of well known biocompatible materials such as woven polyester including that available under the trademark “DACRON”, porous polyurethane, and Polytetrafluroethylene (PTFE). In a preferred embodiment, the biocompatible material is expanded Polytetrafluroethylene (ePTFE). Methods for making ePTFE are well known in art, and are also described in U.S. Pat. No. 4,187,390 issued to Gore on Feb. 5, 1980, which is incorporated herein by reference.

The graft portion 100 can be secured to the rails 80 and the stent portion 220 as illustrated in the U.S. patent application Ser. No. 10/641,284, filed on Aug. 15, 2003, which has been fully incorporated herein by reference. For example, the stent-graft portion 100 can include a plurality of circumferentially extending rings that are spaced from each other along the length of the graft portion 100. These rings eliminate the need to suture the stent portion 200 to the graft portion 100. Additionally, these rings can receive the rails 80 so that the rings and the stent-graft section can move along and relative to the rails 80.

The rails 80 can have any form. For example, the rails 80 can be solid cylindrical members, such as wires or extrusions with circular, elliptical or other known cross sections. Alternatively, the rails 80 can be ribbons or spring wires. Additionally, the rails 80 are desirably sufficiently flexible to accommodate bends, curves, etc. in a blood vessel. Rails 80 may be made from, for example and without limitation the following biocompatible materials: metals, metallic alloys including those discussed above, glass or acrylic, and polymers including bioabsorbable polymers. The rails 80 can also include any of the materials discussed in the U.S. patent application Ser. No. 10/100,986, filed on Mar. 20, 2002, and U.S. Provisional Patent Application Ser. No. 60/426,366, filed on Nov. 15, 2002, which have been incorporated herein by reference.

The rails 80 can be passed or “snaked” through the circumferential support elements 222 as discussed in U.S. patent application Ser. No. 10/641,284. Additionally, the rails 80 can be passed through the stent portion 200 and the graft portion 100 as discussed below.

In the embodiment illustrated in FIGS. 2 and 3, the circumferential support elements 222 include apertures through which the rails 80 extend as shown. The support elements 222 slide along the rail(s) 80 so that the stent-graft assembly 50 can conform to the shape of the aorta or other blood vessel. It is also contemplated that the terminal support elements 222 can move along the rails 80 if, for example, the rail elements form a closed loop or include terminal stop members.

The rail stent assembly 300 includes a plurality of vessel support elements 322 that, like vessel support elements 222, are mounted for free movement along the rails 80 and relative to the rails 80. These vessel support elements can be substantially the same as vessel support elements 222 discussed above. Therefore, the above-discussion regarding vessel support elements 222 is also applicable to vessel support elements 322 and will not be repeated. The adjacent vessel support elements 322 can be secured to each other by a bridge element. Providing at least one bridge element between adjacent support elements 322 increases the structural integrity of the stent-graft 10 because it helps to keep the support elements 322 distributed along the length of the rail stent portion 300 while still offering increased longitudinal flexibility. Alternatively, adjacent vessel support elements 322 can be free of any connection to each other and move independently along the rail(s) 80.

As previously discussed, the rails 80 are desirably sufficiently flexible to accommodate bends, curves, etc. in a blood vessel and can have any of the configurations discussed in U.S. patent application Ser. No. 10/100,986 and U.S. Provisional Patent Application Ser. No. 60/426,366. The ability of the support elements 322 to move along and independent of the rails 80 allows the rail stent section 300 to conform to the contour of a vessel by shortening along the inner radius of a vessel curve and maintaining a longer arc along the outer radius of the vessel curve. This conformability of the rail stent assembly 300 creates an effective seal with the vascular wall of the aorta above the renal artery junction 16. Similarly, as discussed above, the stent-graft assembly 50 is also capable of experiencing this conformability to the shape of the aorta below the junction 16 and thus is capable of forming a seal with the lower part of the descending aorta.

As shown in FIG. 2, the rails 80 extend between the rail stent assembly 300 and the stent-graft assembly 50. The space 90 between the rail stent assembly 300 and the stent-graft assembly 50 is aligned with the junction 16 and formed by stops on the rail(s) 80. Specifically, each rail 80 can include a mechanical deformation or stop member, such as a weld that restrict the support elements 222, 322 from traveling along the rail and entering the open space 90. Alternatively, the rails may be free of any type of stop for either the rail stent assembly 300 and/or the stent-graft assembly 50.

The stent-graft assembly 60 can include a bifurcated region 65 as shown in FIG. 2. In a preferred embodiment, the bifurcated region permits the stent-graft assembly 60 to be used in cases where involvement of one or both iliac vessels 11 and 13 is present. The bifurcated region 65 of the stent-graft 60 has a generally Y-shape and extends from the primary section 62 of the stent-graft assembly 60 that is located within the aorta 12. The bifurcated region includes a first limb 64 for location within a vessel such as the ipsilateral iliac vessel 11, and a second limb 66 for location within another vessel such as the contralateral iliac vessel 13. These limbs 64, 66 meet at a graft limb junction 63. Each limb 64, 66 is generally similar in construction to the primary section 60. Both limbs utilize the rail stent-graft technology discussed above with respect to the primary section 62. For example, the limbs 64, 66 each include a graft portion 100 having a graft material that can be secured relative to a stent portion 200 that includes a plurality of vessel support elements 222. The graft portion 100 and stent portion 200 of each limb 64, 66 are moveable between the ends of the rails 80 that support them. The term “bifurcation” is not limiting to the number of limbs that can found in this region of the stent-graft 10. Instead, the bifurcated region 65 could include more than two limbs.

The present invention also includes introducing an agent into a body using the above-discussed stent-graft 10. In a preferred embodiment, the agent(s) is carried by one or more of the rails 80 or the graft portion 100 and released within the body over a predetermined period of time. For example, these stents can deliver one or more known agents, including therapeutic and pharmaceutical drugs, at a site of contact with a portion of the vasculature system or when released from a carrier as is known. These agents can include any known therapeutic drugs, antiplatelet agents, anticoagulant agents, antimicrobial agents, antimetabolic agents and proteins. These agents can also include any of those disclosed in the above mentioned U.S. Provisional Patent Application No. 60/426,366, U.S. Pat. No. 6,153,252 to Hossainy et al., and U.S. Pat. No. 5,833,651 to Donovan et al., all of which are hereby incorporated by reference in their entirety. Local delivery of these agents is advantageous in that their effective local concentration is much higher when delivered by the stent than that normally achieved by systemic administration.

Thus, while there have been shown and described and pointed out fundamental novel features of the present invention as applied to preferred embodiments thereof, it will be understood that various omissions and substitutions and changes in the form and details of the devices illustrated, and in their operation, and in the method illustrated and described, may be made by those skilled in the art without departing from the spirit of the invention as broadly disclosed herein.

1. A rail stent-graft comprising:
an elongated stent assembly comprising at least one vessel support element and being positionable on a first side of a junction of at least two vessels;
an elongated stent-graft assembly comprising at least one vessel support element and at least one graft element, said stent-graft assembly being positionable on a second side of the junction of the at least two vessels, and
at least one rail element extending between said stent assembly and said stent-graft assembly, each said assembly being moveable along and relative to said at least one rail element.

2. The stent-graft according to claim 1, wherein said graft portion includes circumferential rings spaced along said axis, and wherein said at least one rail element is received in a plurality of said longitudinally spaced rings.

3. The stent-graft according to claim 1, wherein said stent assembly and said stent-graft assembly each include a plurality of vessel support elements.

4. The stent-graft according to claim 3, wherein said vessel support elements are substantially circumferential in cross section.

5. The stent-graft according to claim 1, wherein said graft element is formed of expanded Polytetrafluroethylene.
